(19) United States
(12) Patent Application Publication
(10) Pub. No.: US 2021/0093300 A1
(43) Pub. Date: Apr. 1, 2021

(54) ULTRASONIC DIAGNOSTIC APPARATUS AND ULTRASONIC DIAGNOSTIC METHOD

(71) Applicants: Ryuichi NAKAHARA, (US); Canon Medical Systems Corporation, Otawara-shi (JP)

(72) Inventors: Ryuichi NAKAHARA, Okayama (JP); Keiichiro NISHIDA, Okayama (JP); Toshifumi OZAKI, Okayama (JP); Yoshihisa NASU, Okayama (JP); Tatsuo MAEDA, Nasushiobara (JP)

(73) Assignees: Ryuichi NAKAHARA, Okayama-shi (JP); Canon Medical Systems Corporation, Otawara-shi (JP)

(21) Appl. No.: 17/038,455

(22) Filed: Sep. 30, 2020

(30) Foreign Application Priority Data
Sep. 30, 2019 (JP) 2019-180142

(51) Int. Cl. A61B 8/08; A61B 8/00; G06K 9/62; G06T 7/11

(52) U.S. Cl. CPC A61B 8/5207; A61B 8/0891; G06T 7/11; G06K 9/6218; G06K 9/6215; A61B 8/461

(57) ABSTRACT

According to one embodiment, an ultrasonic diagnostic apparatus includes processing circuitry. The processing circuitry acquires a plurality of ultrasonic images relating to a subject, the plurality of ultrasonic images being obtained by varying an incident direction of an ultrasonic beam. The processing circuitry detects, based on the plurality of ultrasonic images, anisotropies at positions in an ultrasonic image with respect to the ultrasonic beam. The processing circuitry classifies tissues of the subject into regions, based on brightness values of the ultrasonic image and the anisotropies.

### CROSS-REFERENCE TO RELATED APPLICATIONS

This application is based upon and claims the benefit of priority from the prior Japanese Patent Application No. 2019-180142, filed Sep. 30, 2019, the entire contents of which are incorporated herein by reference.

### FIELD

Embodiments described herein relate generally to an ultrasonic diagnostic apparatus and an ultrasonic diagnostic method.

### BACKGROUND

In segmentation of an ultrasonic moving image obtained by an ultrasonic diagnostic apparatus, it is difficult to accurately distinguishing muscles, fat, tendons, blood vessels, and the like from one another and perform segmentation because brightness differences between tissues are small.

### BRIEF DESCRIPTION OF THE DRAWINGS

FIG. 1 is a block diagram of an ultrasonic diagnostic apparatus according to the present embodiment.

FIG. 2 is a flowchart showing a first operation example of the ultrasonic diagnostic apparatus according to the present embodiment.

FIG. 3A shows a first acquisition example of a plurality of ultrasonic images.

FIG. 3B shows a second acquisition example of a plurality of ultrasonic images.

FIG. 4 shows a calculation example of anisotropic curves according to the present embodiment.

FIG. 5 shows a calculation example of anisotropic curves when a plurality of tissues are present in a shallower layer than an anisotropy detection target tissue.

FIG. 6 shows an example of an ultrasonic image that is a result of grouping based on corrected brightness values according to the present embodiment.

FIG. 7 shows an example of a segmentation image according to the present embodiment.

FIG. 8 is a flowchart showing a second operation example of the ultrasonic diagnostic apparatus according to the present embodiment.

FIG. 9 shows an example of clustering processing according to the present embodiment.

### DETAILED DESCRIPTION

In general, according to one embodiment, an ultrasonic diagnostic apparatus includes processing circuitry. The processing circuitry acquires a plurality of ultrasonic images relating to a subject, the plurality of ultrasonic images being obtained by varying an incident direction of an ultrasonic beam. The processing circuitry detects, based on the plurality of ultrasonic images, anisotropies at positions in an ultrasonic image with respect to the ultrasonic beam. The processing circuitry classifies tissues of the subject into regions, based on brightness values of the ultrasonic image and the anisotropies.

Hereinafter, an ultrasonic diagnostic apparatus and an ultrasonic diagnostic method will be described. In the following embodiments, elements assigned the same reference numeral perform the same operation, and redundant descriptions will be omitted as appropriate. Hereinafter, one embodiment will be described with reference to the accompanying drawings.

### First Embodiment

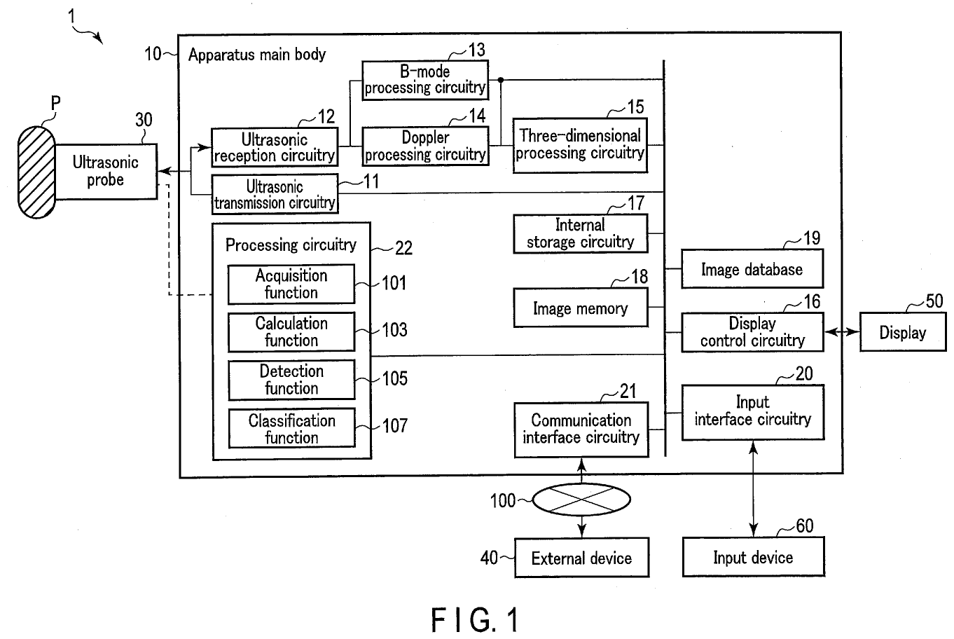

An ultrasonic diagnostic apparatus according to the present embodiment will be described with reference to the block diagram of FIG. 1.

FIG. 1 is a block diagram showing a configuration example of an ultrasonic diagnostic apparatus 1 according to the present embodiment. As shown in FIG. 1, the ultrasonic diagnostic apparatus 1 includes an apparatus main body 10 and an ultrasonic probe 30. The apparatus main body 10 is connected to an external device 40 via a network 100. The apparatus main body 10 is connected to a display 50 and an input device 60.

The ultrasonic probe 30 includes a plurality of ultrasonic transducers (hereinafter also simply referred to as “elements”), a matching layer provided in each element, and a backing material that prevents backward propagation of ultrasonic waves from the elements. The ultrasonic probe 30 is detachably connected to the apparatus main body 10.

The ultrasonic probe 30 according to the present embodiment may be provided with a position sensor so that positional information can be detected when a subject P is three-dimensionally scanned. The ultrasonic probe 30 according to the present embodiment may be, for example, a two-dimensional array probe, in which a plurality of ultrasonic transducers are arranged in a matrix. Alternatively, the ultrasonic probe 30 may be a mechanical four-dimensional probe (mechanical-swinging-type three-dimensional probe) which includes a one-dimensional array probe and a probe swinging motor in an enclosure, mechanically performs a swing scan or a rotary scan by swinging ultrasonic transducers at a predetermined angle (swinging angle), and thereby three-dimensionally scans a subject P. The ultrasonic probe 30 may be a 1.5-dimensional array probe, in which one-dimensionally arranged transducers are divided into a plurality of groups, or a one-dimensional array probe, in which a plurality of ultrasonic transducers are simply aligned in an array direction in a row.

The apparatus main body 10 shown in FIG. 1 generates an ultrasonic image, based on a reflected wave signal received by the ultrasonic probe 30. As shown in FIG. 1, the apparatus main body 10 includes ultrasonic transmission circuitry 11, ultrasonic reception circuitry 12, B-mode processing circuitry 13, Doppler processing circuitry 14, three-dimensional processing circuitry 15, display control circuitry 16, internal storage circuitry 17, an image memory 18 (cine memory), an image database 19, input interface circuitry 20, communication interface circuitry 21, and control circuitry 22.

The ultrasonic transmission circuitry 11 is a processor that supplies a drive signal to the ultrasonic probe 30. The ultrasonic transmission circuitry 11 is implemented by, for example, trigger generation circuitry, delay circuitry, and pulser circuitry. The trigger generation circuitry repeatedly generates a rate pulse for forming transmission ultrasonic waves at a predetermined rate frequency. The delay circuitry provides each rate pulse generated by the trigger generation circuitry with a transmission delay time for each element, which is necessary for converging ultrasonic waves generated by the ultrasonic probe 30 in a beam form and determining a transmission directivity. The pulser circuitry applies a drive signal (drive pulse) to the ultrasonic probe 30 at a timing based on the rate pulses. By varying the transmission delay time provided to each rate pulse by the delay circuitry, the transmission direction from the element surface can be discretionarily adjusted.

The ultrasonic reception circuitry 12 is a processor that performs various types of processing on the reflected wave signal received by the ultrasonic probe 30 and thereby generates a reception signal. The ultrasonic reception circuitry 12 is implemented by, for example, amplifier circuitry, an A/D converter, reception delay circuitry, and an adder. The amplifier circuitry executes gain correction processing by amplifying, for each channel, the reflected wave signal received by the ultrasonic probe 30. The A/D converter converts the gain-corrected reflected wave signal into a digital signal. The reception delay circuitry provides the digital signal with a reception delay time which is necessary for determining a reception directivity. The adder sums a plurality of digital signals each provided with a reception delay time. By the summation processing of the adder, a reception signal with an enhanced reflected component in a direction corresponding to the reception directivity is generated.

The B-mode processing circuitry 13 is a processor that generates B-mode data based on the reception signal received from the ultrasonic reception circuitry 12. The B-mode processing circuitry 13 performs envelope wave detection processing, logarithmic amplification processing, and the like, on the reception signal received from the ultrasonic reception circuitry 12 to generate data (B-mode data) that expresses signal strength by brightness. The generated B-mode data is stored in a raw data memory (not shown) as B-mode raw data on an ultrasonic scanning line. The B-mode raw data may be stored in the internal storage circuitry 17 to be described later.

The Doppler processing circuitry 14 is, for example, a processor, and extracts a blood-flow signal from the reception signal received from the ultrasonic reception circuitry 12, and generates Doppler waves from the extracted blood-flow signal as well as generating data (hereinafter “Doppler data”) obtained by extracting, from the blood-flow signal, information on average velocity, distribution, power, and the like, at multiple points.

The three-dimensional processing circuitry 15 is a processor capable of generating two-dimensional image data or three-dimensional image data (hereinafter also referred to as “volume data”) based on the B-mode data and Doppler data generated by the B-mode processing circuitry 13 and the Doppler processing circuitry 14, respectively. The three-dimensional processing circuitry 15 performs a raw-pixel conversion to generate two-dimensional image data consisting of pixels.

The three-dimensional processing circuitry 15 also performs a raw-voxel conversion including interpolation processing, in which spatial positional information is taken into consideration, on the B-mode raw data stored in the raw data memory to generate volume data consisting of voxels in a desired range. The three-dimensional processing circuitry 15 also performs rendering processing on the generated volume data to generate rendering image data. Hereinafter, the B-mode raw data, the two-dimensional image data, the volume data, and the rendering image data will also be collectively referred to as ultrasonic data.

The display control circuitry 16 converts image data into a video signal by performing various types of processing, such as dynamic range, brightness, contrast, y curve corrections, and an RGB conversion, on various types of image data generated at the three-dimensional processing circuitry 15. The display control circuitry 16 causes the display 50 to display the video signal. The display control circuitry 16 may generate a user interface (graphical user interface: GUI) for an operator to input various instructions through the input interface circuitry 20, and cause the display 50 to display the GUI. As the display 50, for example, a CRT display, a liquid crystal display, an organic EL display, an LED display, a plasma display, or any other display known in the relevant technical field may be used as appropriate.

The internal storage circuitry 17 includes, for example, a magnetic or optical storage medium, or a processor-readable storage medium such as a semiconductor memory. The internal storage circuitry 17 stores, for example, a control program relating to a delay amount setting method according to the present embodiment, a control program for realizing ultrasonic transmission and reception, a control program for performing image processing, and a control program for performing display processing. The internal storage circuitry 17 also stores diagnostic information (such as a patient's ID and a doctor's observation), a diagnostic protocol, a body mark generation program, and a data group such as a conversion table in which the range of color data used for imaging is preset for each diagnostic site. The internal storage circuitry 17 may also store an anatomical picture, such as an atlas, relating to the structures of internal organs in the subject.

The internal storage circuitry 17 also stores the two-dimensional image data, volume data, and rendering image data generated at the three-dimensional processing circuitry 15, in accordance with a storing operation input through the input interface circuitry 20. The internal storage circuitry 17 may store the two-dimensional image data, volume data, and rendering image data generated at the three-dimensional processing circuitry 15 together with the operation order and operation time, in accordance with a storing operation input through the input interface circuitry 20. The internal storage circuitry 17 can also transfer the stored data to an external device via the communication interface circuitry 21.

The image memory 18 includes, for example, a magnetic or optical storage medium, or a processor-readable storage medium such as a semiconductor memory. The image memory 18 stores image data corresponding to a plurality of frames immediately before a freeze operation input through the input interface circuitry 20. The image data stored in the image memory 18 is, for example, sequentially displayed (cine-displayed).

The image database 19 stores image data transferred from the external device 40. For example, the image database 19 receives and stores historical medical image data relating to the same patient, that was obtained in past medical examinations and stored in the external device 40. The historical medical image data includes ultrasonic image data, computed tomography (CT) image data, magnetic resonance (MR) image data, position emission tomography (PET)-CT image data, PET-MR image data, and X-ray image data.

The image database 19 may store desired image data by reading image data stored in a storage medium such as a magneto-optical disk (MO), a CD-R, or a DVD.

The input interface circuitry 20 receives various instructions from an operator through the input device 60. The input device 60 is, for example, a mouse, a keyboard, a panel switch, a slider switch, a trackball, a rotary encoder, an operation panel, or a touch command screen (TCS). The input interface circuitry 20 is connected to the processing circuitry 22 via, for example, a bus, converts an operation instruction input by the operator into an electrical signal, and outputs the electrical signal to the processing circuitry 22. Herein, the input interface circuitry 20 is not limited to circuitry connected to a physical operational component, such as a mouse or a keyboard. Examples of the input interface circuitry 20 include processing circuitry of an electrical signal, which receives, as a radio signal, an electrical signal corresponding to an operation instruction input through an external input device provided separately from the ultrasonic diagnostic apparatus 1 and outputs the electrical signal to the control circuitry 22. The external input device may be, for example, an external input device capable of transmitting, as a radio signal, an operation instruction corresponding to an instruction corresponding to an operator's gesture.

The communication interface circuitry 21 is connected to the external device 40 via, for example, the network 100, and performs data communication with the external device 40. The external device 40 is, for example, a database of a picture archiving and communication system (PACS), which is a system that manages various types of medical image data, and a database of an electronic health record system, which manages electronic health records accompanied by medical images. The external device 40 may also be any medical image diagnostic apparatus other than the ultrasonic diagnostic apparatus 1 according to the present embodiment, such as an X-ray CT apparatus, a magnetic resonance imaging (MRI) apparatus, a nuclear medicine diagnostic apparatus, or an X-ray diagnostic apparatus. The standard of communication with the external device 40 may be any standard, but is, for example, digital imaging and communications in medicine (DICOM).

The processing circuitry 22 is, for example, a processor that functions as a nerve center of the ultrasonic diagnostic apparatus 1. The processing circuitry 22 executes a control program stored in the internal storage circuitry 17, thereby implementing a function corresponding to the program.

The processing circuitry 22 includes an acquisition function 101, a calculation function 103, a detection function 105, and a classification function 107.

Through the acquisition function 101, the processing circuitry 22 acquires a plurality of ultrasonic images relating to a subject, which are obtained by varying the ultrasonic beam incident direction.

Through the calculation function 103, the processing circuitry 22 calculates a corrected pixel value obtained by correcting an attenuation amount of an ultrasonic beam (also simply referred to as a beam) based on at least one ultrasonic image. Instead of merely calculating the corrected pixel value, the processing circuitry 22 may generate, through the calculation function 103, a corrected attenuation amount image, in which the attenuation amount of the beam has been corrected, based on the corrected pixel value.

Through the detection function 105, the processing circuitry 22 detects an anisotropy of a tissue of the subject with respect to the beam, based on the ultrasonic images. Anisotropy means that a body tissue included in a subject exhibits different signal values or brightness values depending on the incident direction of a beam to the body tissue, or the degree of the property. The anisotropy may be evaluated for example by the shape of the change curve (hereinafter also referred to as an “anisotropic curve”) of the signal value or brightness value with respect to the beam incident direction or an index value corresponding to the shape.

Through the classification function 107, the processing circuitry 22 classifies tissues of the subject based on brightness values and anisotropies of the ultrasonic images.

The acquisition function 101, calculation function 103, detection function 105, and classification function 107 may be incorporated in the processing circuitry 22 or the apparatus main body 10 as control programs or as dedicated hardware circuits capable of performing respective functions.

The processing circuitry 22 may be implemented by an application specific integrated circuit (ASIC), a field programmable gate array (FPGA), or another complex programmable logic device (CPLD) or simple programmable logic device (SPLD), into which such dedicated hardware circuitry is incorporated.

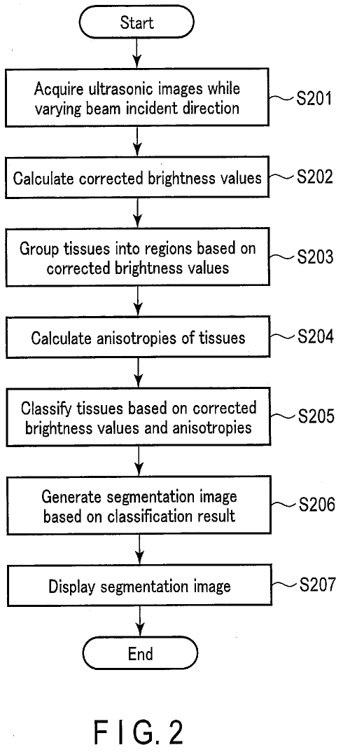

A first operation example of the ultrasonic diagnostic apparatus 1 according to the present embodiment will be described with reference to the flowchart of FIG. 2.

In step S201, through the acquisition function 101, the processing circuitry 22 acquires a plurality of ultrasonic images relating to a subject obtained by medical staff scanning the subject P with the ultrasonic probe 30, and varying the incident direction of the beam into the body of the subject P.

In step S202, through the calculation function 103, the processing circuitry 22 calculates corrected brightness values of tissues in a plurality of ultrasonic images acquired for respective beam incident directions, and generates a corrected attenuation amount image. Since the intensity of the beam attenuates as the beam passes through tissues, the brightness value (pixel value) of the B-mode image is lower (i.e., the image becomes darker) in a deeper portion of the image. Accordingly, a tissue in a shallow layer (a layer close to the body surface), which provides a large attenuation and is darkly depicted, and a tissue in a deep layer (a layer deep inside the body far from the body surface), which provides a small attenuation but is darkly depicted because the attenuation amount in the path to the site is large because the tissue is in the deep layer, may be classified into the same group.

Therefore, for a tissue in a deep layer which is a corrected brightness value calculation target, a corrected brightness value obtained by compensating for the beam attenuation amount of a shallow layer which is closer to the body surface than the deep layer, is calculated. As a specific method for calculating the corrected brightness value, the influence of the attenuation amount of a tissue closer to the body surface than the calculation target tissue is compensated for; therefore, the correction amount may be manually designated. For example, the correction amount may be designated by adjustment through a slide bar, such as time gain compensation (TGC) and sensitivity time control (STC).

Alternatively, the corrected brightness value of the calculation target tissue may be calculated by sequentially calculating the brightness values of tissues, i.e., the attenuation amounts of an oscillating beam, from the tissue in the shallowest layer to tissues in deeper layers with respect to the beam, and then summing the attenuation amounts.

In step S203, through the calculation function 103 or classification function 107, the processing circuitry 22 groups tissues of the subject P shown in an ultrasonic image into a plurality of regions in each of which the degree of similarity between corrected brightness values is greater than or equal to a threshold, based on the corrected brightness values calculated in step S202. For example, tissues with corrected brightness values within a threshold range are grouped together as a group of regions in which the degree of similarity between corrected brightness values is greater than or equal to a threshold. At this time, a directional pattern or repetitive pattern may be detected by texture analysis, and grouping may be performed based on the directional pattern and repetitive pattern and the corrected brightness values within the threshold range. As a result, through the calculation function 103 or the classification function 107, the processing circuitry generates a corrected attenuation amount image including the regions created by grouping in step S203.

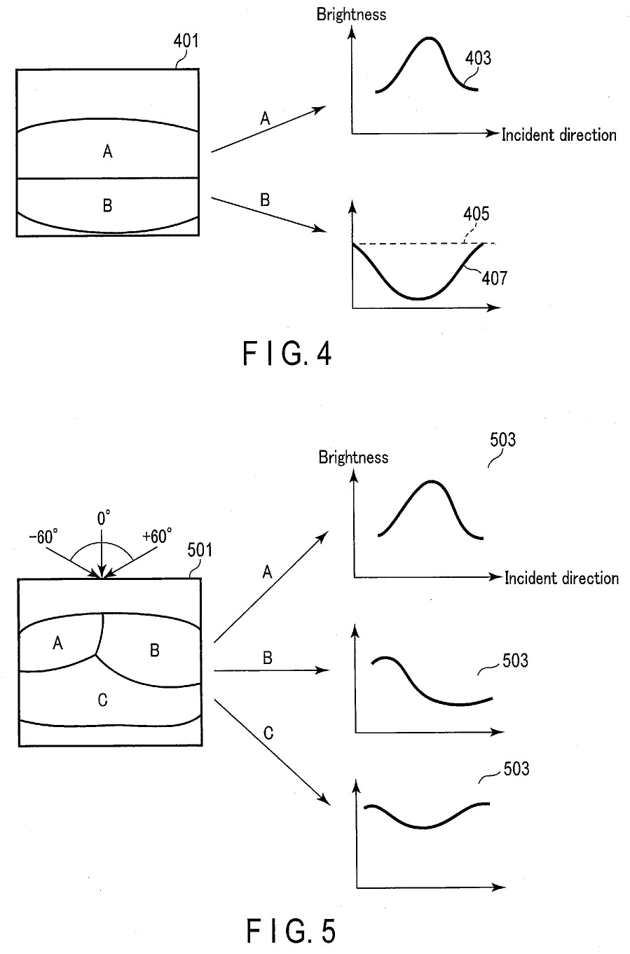

In step S204, through the detection function 105, the processing circuitry 22 calculates anisotropies of tissues of the subject P in the ultrasonic image for each region created by grouping in step S203. To calculate an anisotropy, for example, an anisotropic curve indicating how the brightness value (signal strength) changes in accordance with the change in the beam incident direction is calculated. The anisotropy of a tissue in a deep layer far from the body surface is influenced by the anisotropy of a tissue in a shallow layer close to the body surface. Therefore, the influence of the anisotropy of a tissue in a shallow layer closer to the body surface than the layer of the anisotropy calculation target tissue is compensated for. Specifically, the anisotropy curve of a tissue in a shallow layer is determined, and anisotropies of tissues in deeper layers are sequentially calculated from the layer next to the shallow layer so that the influences of the anisotropic curves of tissues in the shallower layers are compensated for. Details will be described later with reference to FIGS. 4 and 5.

In step S205, through the classification function 107, the processing circuitry 22 classifies tissues to be shown in an ultrasonic image based on the corrected brightness values and anisotropies. For example, through the classification function 107, the processing circuitry 22 groups tissues in each region created by grouping based on the corrected brightness values in step S203 into regions with a degree of similarity between anisotropies greater than or equal to a threshold. Specifically, tissues are classified by grouping together tissues with anisotropic curves having similar shapes.

In step S206, through the classification function 107, the processing circuitry 22 generates a segmentation image in which tissues included in the ultrasonic image are classified based on a result of the classification of tissues.

In step S207, the display control circuitry 16 causes the display 50 to display the segmentation image.

In the classification based on the brightness values calculated in step S203 and the classification based on the anisotropies calculated in step S205, even if regions are separated on an image, the regions are considered to have the same property as long as the degrees of similarity between brightness values and those between anisotropies in a plurality of ultrasonic images are greater than or equal to a threshold, and may be grouped as one group. The grouping processing using brightness values in step S203 and the grouping processing using anisotropies in step S205 may be transposed.

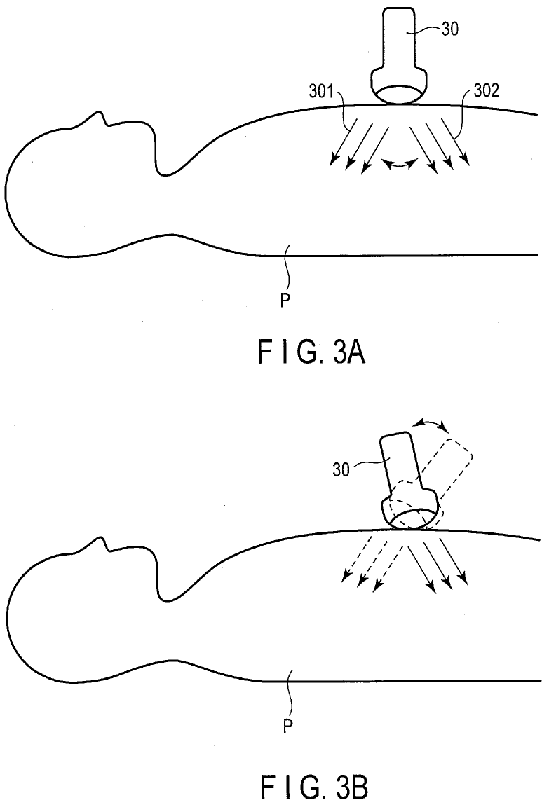

Next, acquisition examples of a plurality of ultrasonic images will be described with reference to FIGS. 3A and 3B. FIG. 3A shows a first acquisition example of ultrasonic images, in which the ultrasonic probe 30 can electronically control a beam. The ultrasonic probe 30 can electronically swing a beam with the contact position of the ultrasonic probe 30 fixed on the body surface of the subject P.

FIG. 3B shows a second acquisition example of ultrasonic images, in which an operator swings the ultrasonic probe 30 for scanning while bringing the ultrasonic probe 30 into contact with the body surface of the subject P. Accordingly, imaging can be performed with different beam incident directions with respect to a tissue of the subject P. By varying the beam incident direction as shown in FIGS. 3A and 3B, anisotropies of tissues in the imaging range of the subject are shown in the ultrasonic images.

To acquire a plurality of ultrasonic images of different beam incident directions for an imaging target range, it should be noted that the imaging target range must be larger than the scanning range. This is because a beam with an angle that enables the beam to enter from outside the scanning range into the scanning range is required. For example, whether or not an ultrasonic image of a beam incident direction necessary for the classification processing according to the present embodiment has been acquired may be calculated based on the coordinate information obtained by the position sensor attached to the ultrasonic probe 30. For example, ultrasonic images of incident directions which have not been acquired yet can be easily recognized by displaying a graph of beam incident directions necessary for the imaging target range, and causing the display to show a GUI on which the incident directions in which the operator has performed scanning are filled in.

Next, a calculation example of anisotropic curves in step S204 will be described with reference to FIG. 4.

On the ultrasonic image 401 in FIG. 4, a tissue A is an area closest to the body surface (shallow layer) and a tissue B is an area farther from the body surface than the tissue A (deep layer). The tissues A and B of the subject P have already been distinguished for convenience of explanation; in practice, however, the tissues may be distinguished by sequentially determining anisotropic curves from the shallow layer to the deep layer.

Here, for each of the tissues A and B, an anisotropic curve 403 of the case where the beam incident direction varied is calculated. The vertical axis of the anisotropic curve 403 indicates the brightness value (signal strength), and the horizontal axis indicates the beam incident direction. As described above, the tissue B in a deeper layer than the tissue A is influenced by the anisotropic curve of the tissue A, which is in a shallower layer than the tissue B in the beam incident direction. For example, let us suppose a case where the brightness value of the tissue A shows an anisotropic curve that is convex upward in accordance with changes in the beam incident direction, and the brightness value of the detection target tissue B is constant regardless of the incident direction, as shown by the anisotropic curve 405 with a broken line. In this case, the actual anisotropic curve of the tissue C is a difference between the curve 405 and the anisotropic curve of the tissue A, and is considered to be a curve 407 that is convex downward.

By sequentially determining anisotropic curves from the tissue A in the shallow layer to the part deep inside the body as described above, correct anisotropic curves can also be calculated for tissues in deep layers with the influences of anisotropies of tissues in shallower layers compensated for.

Next, the case where a plurality of tissues are included in a shallower layer than the anisotropy detection target tissue will be described with reference to FIG. 5.

FIG. 5 is similar to FIG. 4, but shows the case where the tissues A and B are in the shallowest layer closest to the body surface, and the tissue C is in a deeper layer than the tissues A and B.

First, through the detection function 105, the processing circuitry 22 calculates an anisotropic curve 503 of each of the tissues A and B. As described above, the tissue C in a deeper layer than the tissues A and B is influenced by the anisotropic curves of the tissues A and B, which are in a shallower layer than the tissue C in the beam incident direction.

When the anisotropy of the detection target tissue C is detected, the anisotropic curve of the tissue C may be calculated in a manner similar to the case of FIG. 4 while using the average of the anisotropic curve of the tissue A and that of the tissue B as an anisotropic curve of a tissue in a shallower layer than the tissue C.

As the beam incident direction changes, the ratio between the tissues A and B along a straight line in the beam incident direction, which influence the tissue C, changes. For example, as shown in FIG. 5, when a beam enters at the angle of minus 60 degrees, the tissue A is stacked more than the tissue B on the tissue C, whereas, when a beam enters at the angle of plus 60 degrees, the tissue B is stacked more than the tissue A on the tissue C. Therefore, a sum of a weighted anisotropic curve of the tissue A and that of the tissue B may be calculated as an anisotropic curve of a tissue in a shallower layer than the tissue C.

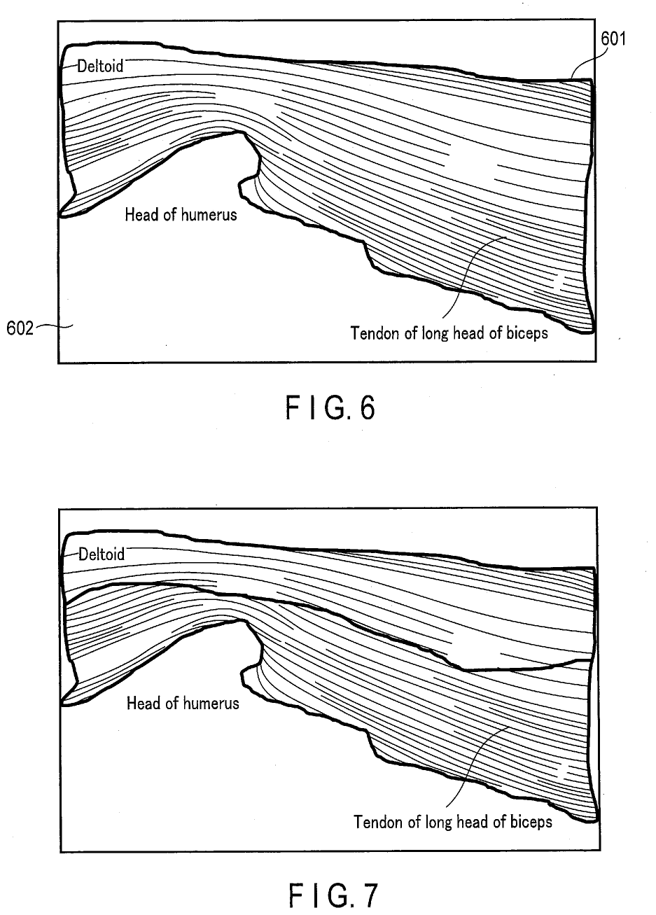

Next, an example of the result of the classification according to the first operation example of the ultrasonic diagnostic apparatus 1 will be described with reference to FIGS. 6 and 7.

FIG. 6 shows, on an ultrasonic image, a result of grouping based on brightness values obtained in step S203. Here, the tissues are classified into a region (region 601) with brightness values greater than or equal to a threshold and a region (region 602) with brightness values smaller than the threshold. The region 601 shows a directional pattern of muscle fibers; therefore, a region with the directional pattern of muscle fibers is judged based on the geometric structure even if the region includes a portion with a brightness value smaller than the threshold. In the classification based on corrected brightness values, the “deltoid” and “tendon of the long head of the biceps” are classified into the same group.

FIG. 7 shows an example of a segmentation image obtained by grouping based on anisotropic curves. In the example, a result of the grouping based on anisotropies is further shown on the ultrasonic image in the region 601 grouped based on corrected brightness values.

As shown in FIG. 7, a segmentation image in which the tissues classified as the same region based on corrected brightness values have been further classified into the “deltoid” and “tendon of the long head of the biceps” can be displayed. In the segmentation image, groups into which tissues are classified may be shown, for example, on a color map in respective colors.

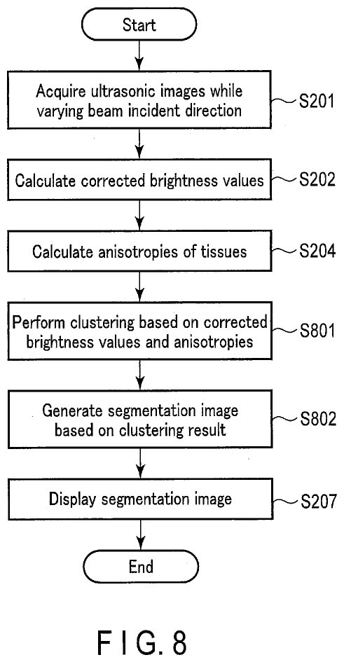

Next, a second operation example of the ultrasonic diagnostic apparatus 1 according to the present embodiment will be described with reference to the flowchart of FIG. 8.

In the first operation example shown in FIG. 2, grouping based on brightness values and grouping based on anisotropies are performed in order. In the second operation example, tissues are classified by clustering using both the brightness value and anisotropy as parameters. Since steps S201, S202, S204, and S207 are the same as those in the first operation example, descriptions of those steps are omitted. As shown in FIG. 8, the operation is performed in the order of step S201, step S202, step S204, step S801, step S802, and step S207.

In step S801, through the classification function 107, the processing circuitry 22 performs clustering based on the brightness values and anisotropies.

In step S802, through the classification function 107, the processing circuitry 22 generates a segmentation image in which tissues included in the ultrasonic image are classified based on a result of the clustering.

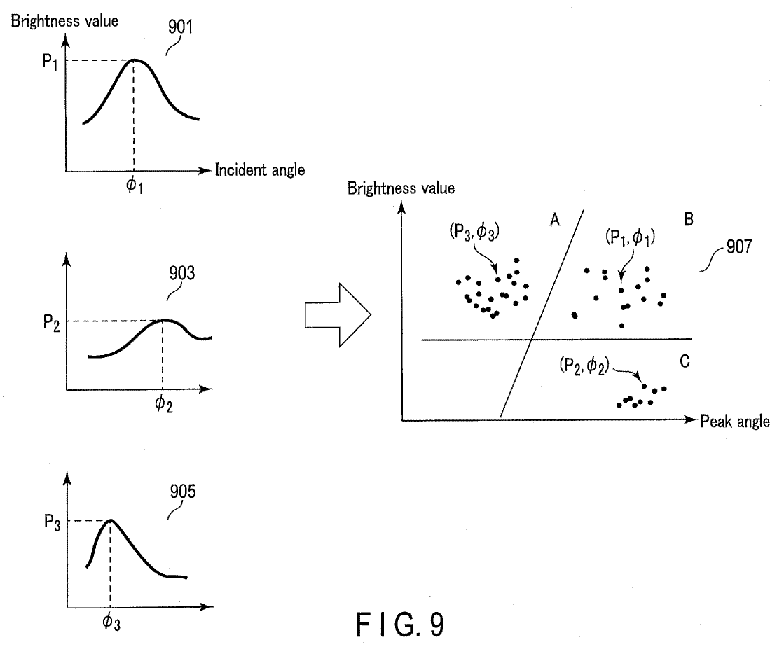

Next, an example of the clustering processing in step S801 will be described with reference to FIG. 9.

The left part of FIG. 9 shows anisotropic curves 901, 903, and 905 of three regions, in each of which the vertical axis indicates the corrected brightness value and the horizontal axis indicates the peak angle, i.e., the beam incident angle when the corrected brightness value is maximum.

When such brightness values and incident angles relating to the anisotropic curves 901, 903, and 905 are plotted as (Pn, φn) (where n=1, 2, and 3) on a coordinate system, a distribution map 907 as shown at the right part of FIG. 9 for example can be obtained.

On the distribution map 907, a set (cluster) of plots of similar brightness values and incident angles can be obtained; therefore, a borderline for classifying plots into clusters can be drawn by common clustering processing, and tissue characteristics based on tissues' anisotropies can be classified into various patterns, such as pattern A, pattern B, and pattern C.

In the segmentation image, tissue regions classified into clusters, i.e., groups, may be shown on a color map in respective colors.

In the above-described grouping processing and clustering processing, the anisotropy is exhibited by one parameter; however, the anisotropy is not limited to this, and may be exhibited by two parameters when clustering processing is performed three-dimensionally. For example, clustering processing may be performed by plotting the parameters of the observation points in a three-dimensional space consisting of the brightness value, first anisotropic parameter, and second anisotropic parameter. Clustering processing may also be performed on a four-or-higher dimensional space by further increasing the number of parameters.

Alternatively, a trained model may be constructed by training a multi-layer network, such as a deep convolutional neural network (DCNN), using training data that takes a plurality of ultrasonic images imaged in a plurality of beam incident directions as input data and outputs a tissue classification result as truth data.

The use of the trained model enables generation of tissue classification result and a segmentation image at a higher speed and with a higher accuracy. It can also reduce the number of ultrasonic images of different beam incident directions necessary for obtaining the tissue classification result.

According to the above-described present embodiment, tissues are classified by brightness value and anisotropy of the tissue based on a plurality of ultrasonic images of different ultrasonic beam incident directions, which enables classification of tissues that cannot be distinguished based only on the brightness values, thereby improving accuracy of tissue classification. In addition, medical staff only has to acquire a plurality of ultrasonic images by slightly expanding the scanning range, and does not need to perform complicated operations. Therefore, there is no influence on the workflow or the like.

Moreover, the functions described in connection with the above embodiment may be implemented, for example, by installing a program for executing the processing in a computer, such as a workstation, etc., and expanding the program in a memory. The program that causes the computer to execute the processing can be stored and distributed by means of a storage medium, such as a magnetic disk (a hard disk, etc.), an optical disk (CD-ROM, DVD, etc.), and a semiconductor memory.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising processor circuitry configured to:
acquire a plurality of ultrasonic images relating to a subject, the plurality of ultrasonic images being obtained by varying an incident direction of an ultrasonic beam;
detect, based on the plurality of ultrasonic images, anisotropies at positions in an ultrasonic image with respect to the ultrasonic beam; and
classify tissues of the subject into regions, based on brightness values of the ultrasonic image and the anisotropies.

2. The ultrasonic diagnostic apparatus according to claim 1, wherein the processing circuitry classifies tissues corresponding to muscles of the subject.

3. The ultrasonic diagnostic apparatus according to claim 1, wherein the processing circuitry classifies the tissues of the subject by grouping together the regions between which a degree of similarity in brightness values and anisotropies is greater than or equal to a threshold.

4. The ultrasonic diagnostic apparatus according to claim 1, wherein the processing circuitry detects an anisotropy of an anisotropy detection target tissue by compensating for an influence of an anisotropy of a tissue closer to a body surface than the anisotropy detection target tissue.

5. The ultrasonic diagnostic apparatus according to claim 1, wherein the processing circuitry is further configured to calculate a brightness value of a brightness value calculation target by compensating for an influence of an attenuation amount of the ultrasonic beam in a tissue closer to a body surface than the brightness value calculation target.

6. The ultrasonic diagnostic apparatus according to claim 1, wherein the processing circuitry is further configured to cause a display to display a segmentation image in which the tissues of the subject are classified.

7. The ultrasonic diagnostic apparatus according to claim 1, wherein the processing circuitry classifies the tissues by clustering based on the brightness values and anisotropies.

8. An ultrasonic diagnostic method, comprising:
acquiring a plurality of ultrasonic images relating to a subject, the plurality of ultrasonic images being obtained by varying an incident direction of an ultrasonic beam;
detecting, based on the plurality of ultrasonic images, anisotropies at positions in an ultrasonic image with respect to the ultrasonic beam; and
classifying tissues of the subject into regions, based on brightness values of the ultrasonic image and the anisotropies.

9. The ultrasonic diagnostic method according to claim 8, wherein the classifying further classifies tissues corresponding to muscles of the subject.

10. The ultrasonic diagnostic method according to claim 8, wherein the classifying classifies the tissues of the subject by grouping together the regions between which a degree of similarity in brightness values and anisotropies is greater than or equal to a threshold.

11. The ultrasonic diagnostic method according to claim 8, wherein the detecting detects an anisotropy of an anisotropy detection target tissue by compensating for an influence of an anisotropy of a tissue closer to a body surface than the anisotropy detection target tissue.

12. The ultrasonic diagnostic method according to claim 8, further comprising calculating a brightness value of a brightness value calculation target by compensating for an influence of an attenuation amount of the ultrasonic beam in a tissue closer to a body surface than the brightness value calculation target.

13. The ultrasonic diagnostic method according to claim 8, further comprising displaying a segmentation image in which the tissues of the subject are classified.

14. The ultrasonic diagnostic method according to claim 8, wherein the classifying classifies the tissues by clustering based on the brightness values and anisotropies.
